(19) United States
(12) Patent Application Publication
(10) Pub. No.: US 2021/0151179 A1
(43) Pub. Date: May 20, 2021

(54) WEARABLE DEVICE AND IOT NETWORK FOR PREDICTION AND MANAGEMENT OF CHRONIC DISORDERS

(71) Applicant: Rajlakshmi Dibyajyoti Borthakur, Bangalore (IN)

(72) Inventor: Rajlakshmi Dibyajyoti Borthakur, Bangalore (IN)

(21) Appl. No.: 16/636,037

(22) PCT Filed: Aug. 3, 2018
(86) PCT No.: PCT/IN2018/050509

§ 371 Date: Feb. 3, 2020

(30) Foreign Application Priority Data
Aug. 3, 2017 (IN) 201741004103

(51) Int. Cl. G16H 40/67; A61B 5/0205; A61B 5/00; A61B 5/16; G06Q 50/00; G16H 10/60; G16H 50/70; G16H 15/00; G16H 40/20; G06Q 40/08; G16H 10/20; G16H 50/20; G16H 50/50; G16H 20/10; G16H 20/70; G16Y 10/75; G16Y 20/40; G16Y 40/10

(52) U.S. Cl. CPC G16H 40/67; A61B 5/021; A61B 5/0022; A61B 5/7275; A61B 5/165; A61B 5/486; A61B 5/4848; A61B 5/7203; G06Q 50/01; G16H 10/60; G16H 50/70; G16H 15/00; G16H 40/20; G06Q 40/08; G16H 10/20; G16H 50/20; G16H 50/50; G16H 20/10; G16H 20/70; G16Y 10/75; G16Y 20/40; G16Y 40/10; A61B 5/02055

(57) ABSTRACT

A computing system adapted for prediction and management of chronic disorders implemented in an Internet of Things (IoT) network environment is described. The computing system includes an input module to receive a plurality of inputs, the plurality of inputs comprising wearable device sensor inputs indicative of physiological parameters of an individual, distributed IoT system inputs indicative of additional physiological parameters of the individual and surroundings of the individual, enterprise system inputs indicative of medical information of the individual, social media inputs indicative of sentiments of the individual, and user inputs provided by the individual. A processing module performs multimodal, multisource, and multilingual processing on the plurality of inputs to generate a profile of the individual, identify patterns, determine triggers, stressors, reaction, and recovery, and predict an adverse event. A display module provides a report based on the processing and provide an alert when the adverse event is predicted.

### TECHNICAL FIELD

The present subject matter relates, in general, to a wearable device, and, in particular, to an Internet of Things (IoT)-powered wearable device for prediction and management of chronic disorders.

### BACKGROUND

Health care monitoring has become an important part of the practice of medicine. Health care providers need condensed and specific information on patients to provide improved treatment. For instance, the health care providers require condensed and specific information for monitoring health of individuals or patients in institutions or at home to effectively and properly diagnose and treat various chronic diseases, for example, epileptic seizures. During a seizure, a patient is usually unable to get help, talk, think, or act. In some instances, it is very important for doctors and/or caregivers to be able to detect seizures and give the patient immediate help. Patients may suffer related injuries, such as from falls, traffic accidents, and other events. There are some types of seizures, if not attended to, that can be fatal. Continuous monitoring of patient's health, like those of people with seizures, may be required to provide predictions before occurrence of an adverse event.

### BRIEF DESCRIPTION OF DRAWINGS

The detailed description is described with reference to the accompanying figures. In the figures, the left-most digit(s) of a reference number identifies the figure in which the reference number first appears. The same numbers are used throughout the drawings to reference like features and components.

FIG. 1 illustrates a block diagram of a wearable device embedded with a plurality of sensors for prediction and management of chronic disorders, in accordance with an example embodiment of the present subject matter.

FIG. 2 illustrates a health prediction system implemented with the wearable device of FIG. 1, in accordance with an example embodiment of the present subject matter.

FIG. 3 illustrates a block diagram of the health prediction system of FIG. 2, in accordance with an example embodiment of the present subject matter.

FIG. 4 illustrates another block diagram of the health prediction system of FIG. 2, in accordance with an example embodiment of the present subject matter.

FIG. 5 illustrates an example process for health prediction, in accordance with an example embodiment of the present subject matter.

### DETAILED DESCRIPTION

The subject matter described herein relates to an Internet of Things (IoT)-powered wearable device and an IoT network that includes various other devices and sensors for the prediction and management of chronic disorders. In particular, the wearable device and IoT network may be adapted to extract and process data related to an individual's health to monitor physiological parameters non-invasively on a continuous basis for an individual with or without a chronic disorder and provides predictions prior to the occurrence of an adverse event. Based on the predictions, preventive care can be provided to the individual, and the adverse events either aborted, arrested, or managed in an appropriate manner, by either caregivers or medical professionals.

Health care providers may require condensed and specific information on patients or individuals for remote monitoring, diagnosing, and treating the patients or the individuals suffering from disorders such as central nervous system (CNS) related disorders, cardiological disorders, psychological disorders, and orthopedic disorders. For instance, to properly diagnose and treat epileptic seizures, continuous monitoring of the patient's status may be required.

Conventionally, devices are limited by the type of data collected, portability, and nature of data processing that is performed. For example, there are electroencephalography (EEG) machines, which measure electrical neurological activity and are traditionally used to detect seizures. However, EEGs are for hospital use and the hardware is large and expensive. They also involve wired probes being placed on patients' head, making mobility a challenge. Data given by EEGs also need to be interpreted manually by trained personnel, such as technicians and doctors. However, wearable sensors can now provide similar information pertaining to seizures in a more compact and useable form factor.

Further, it is known that every individual has certain patterns of biomedical or physiological signals representing health of the corresponding individual during normal circumstances. It is observed that in conditions like epilepsy, the signals start showing abnormal patterns. However, the abnormal patterns may be individual specific, i.e., different individuals may show different abnormal patterns. Some of the conventional devices like EEG or wearable devices may not be able to differentiate accurately between a normal condition and an abnormal condition. This may be because of a limitation of the parameters used by the conventional and wearable devices.

The present subject matter enables the creation of unique and personalized profiles using biomedical signal data collected from device sensors along with data from other sources. Data from sensors is automatically infused with input from third party IoT and other static and internet-enabled electronic systems, large enterprise systems like electronic medical reports (EMR), hospital management systems, social media systems, global positioning system (GPS), environment-related data, and other smart systems to generate personal health profiles and predictive health insights about individuals. In addition, user-input data may also be added along with the input from various sensors. The unique profiles thus created are updated and adjusted automatically by the wearable system as it collects more data over a period of time.

In one example, input may be collected from one or more device sensors. The parameters collected by wearable device sensors may comprise the following or additional parameters as illustrated in Table 1.

TABLE 1
Parameters measured by sensors

| Parameter Name | Description |
|---|---|
| Skin temperature | The skin temperature is the surface |
| | temperature of the skin. This could |
| | be an analog sensor integrated with |
| | the wearable. This could be an analog |
| | or digital sensor integrated with |
| | the wearable. |
| Body temperature | Body temperature is a measure of |
| | the body's ability to generate |
| | and get rid of heat. This could |
| | be an analog sensor integrated |
| | with the wearable. This could be |
| | an analog or digital sensor |
| | integrated with the wearable. |
| Heart Rate (HR) | The number of heartbeats per unit |
| | of time, usually per minute. This |
| | could be a digital or optical sensor |
| | integrated with the wearable. This |
| | could be an analog, digital or IR |
| | sensor integrated with the wearable. |
| Heart rate variability | HRV is the physiological phenomenon |
| (HRV) | of variation in the time interval |
| | between heartbeats. It is measured |
| | by the variation in the beat-to-beat |
| | interval. This is derived from the |
| | HR sensor. |
| Standard Deviation of | The standard deviation of all normal |
| Normal to Normal | R-R intervals, calculated over a |
| Intervals (SDNN) | 24-hour period and reported in units |
| | of ‘milliseconds’. This is derived |
| | from the HR sensor. |
| the Standard | SDANN is therefore a measure of |
| Deviation of the | changes in heart rate due to cycles |
| Average of NN | longer than 5 minutes. This is |
| Intervals (SDANN) | calculated over short periods, |
| | usually 5 minutes of a 24-hour |
| | recording. This is derived from the |
| | HR sensor. |
| SDNN Index & | Two variants of the SDNN, created by |
| SDANN Index | dividing the 24-hour monitoring period |
| | into 5-minute segments, are the SDNN |
| | index and the SDANN index (both with |
| | units in milliseconds). The SDNN index |
| | is defined as the mean of all the 5- |
| | minute standard deviations of NN |
| | (normal RR) intervals during the 24- |
| | hour period (i.e., the mean of 288 NN |
| | standard deviations), while the SDANN |
| | index is defined as the standard |
| | deviation of all the 5-minute NN |
| | interval means (i.e., the standard |
| | deviation of 288 NN means). This is |
| | derived from the HR sensor. |
| Root Mean Square of | The square root of the mean of the |
| the Successive | squares of the successive differences |
| Differences (RMSSD) | between adjacent NNs. This is derived |
| | from the HR sensor. |
| Total Power | Total power can be defined as the total |
| | spectral power of the NN interval in |
| | the range of frequencies between 0 and |
| | 0.4 Hz. This is derived from the HR sensor. |
| Ultra-low Frequency | Ultralow frequency is defined as the |
| | total spectral power of the NN interval |
| | in the range of frequencies between 0 |
| | and 0.003 Hz. of a 24-hour recording. |
| | This is derived from the HR sensor. |
| Very low Frequency | Very low frequency is defined as the |
| | total spectral power of the NN interval |
| | in the range of frequencies between |
| | 0.003 and 0.04 Hz. This is derived from |
| | the HR sensor. |
| Low Frequency | Low frequency is defined as the total |
| | spectral power of the NN interval in |
| | the range of frequencies between 0.04 |
| | and 0.15 Hz. This is derived from the |
| | HR sensor. |
| High Frequency | High frequency is defined as the total |
| | spectral power of the NN interval in |
| | the range of frequencies between 0.15 |
| | and 0.4 Hz. This is derived from the |
| | HR sensor. |
| LF/HF Ratio | LF/HF ratio is defined as the ratio |
| | between the power of low frequency |
| | and high frequency bands. This is |
| | derived from the HR sensor. |
| Normalized Low | Normalized low frequency is the |
| Frequency | ratio between absolute (i.e., positive) |
| | value of the low frequency and difference |
| | between total power and very low F- |
| | requency and is calculated in percentile. |
| | This is derived from the HR sensor. |
| Normalized High | Normalized High Frequency is the |
| Frequency | ratio between absolute (i.e., positive) |
| | value of the high frequency and difference |
| | between total power and very low frequency |
| | and is calculated in percentile. This is |
| | derived from the HR sensor. |
| BP trend | A pattern of gradual change in the blood |
| | pressure, represented by a line or curve |
| | on a graph. This is derived from the |
| | HR sensor. |
| Respiration trend | A pattern of gradual change in the |
| | respiration, represented by a line or |
| | curve on a graph. This is derived from |
| | an analog or digital HR or Respiration |
| | sensor. |
| SpO2 | SpO2 is an estimate of arterial oxygen |
| | saturation, or SaO2, which refers to |
| | the amount of oxygenated haemoglobin |
| | in the blood. This is derived from the |
| | HR sensor. |
| EDA | The change in the electrical activity |
| | of the skin. This could be an analog |
| | sensor integrated with the wearable. |
| | This is derived from the EDA sensor. |
| Skin Conductance | Tonic level of electrical conductivity |
| Level (SCL) | of skin. This is derived from the |
| | EDA sensor. |
| Change in SCL | Gradual changes in SCL measured at |
| | two or more points in time. This is |
| | derived from the EDA sensor. |
| Frequency of NS- | Number of SCRs in absence of |
| SCRs (nonspecific | identifiable eliciting stimulus. |
| skin conductance | This is derived from the EDA sensor. |
| response) | |
| Skin Conductance | Phasic increase in conductance |
| Response (SCR) | shortly following stimulus onset. |
| amplitude | This is derived from the EDA sensor. |
| SCR latency | Temporal interval between stimulus |
| | onset and SCR initiation. This is |
| | derived from the EDA sensor. |
| SCR rise time | Temporal interval between SCR |
| | initiation and SCR peak. This is |
| | derived from the EDA sensor. |
| SCR half recovery | Temporal interval between SCR peak |
| time | and point of 50% recovery of SCR |
| | amplitude. This is derived from the |
| | EDA sensor. |
| SCR habitation (trials | Number of stimulus presentations |
| to habituation) | before two or three trials with |
| | no response. This is derived from |
| | the EDA sensor. |
| SCR habituation | Rate and change of ER-SCR amplitude. |
| (slope) | This is derived from the EDA sensor. |
| Electromyography | EMG is used for understanding |
| (EMG) | the electrical activity produced by |
| | skeletal muscles. This could be an |
| | analog sensor integrated with the |
| | wearable. This is derived from |
| | the EMG sensor. |
| Power Spectral | PSD is a measure of a signal's |
| Density (PSD) | power intensity in the frequency |
| | domain. In practice, the PSD is |
| | computed from the FFT spectrum of a |
| | signal. This is derived from the |
| | EMG sensor. |
| Histogram | Used to identify trends within |
| | the EMG data. This is derived from |
| | the EMG sensor. |
| Frequency Domain - | MNF is an average frequency which |
| Mean Power | is calculated as the sum of product |
| Frequency (MNF) | of the EMG power spectrum and the |
| | frequency divided by the total sum |
| | of the power spectrum. This is derived |
| | from the EMG sensor. |
| Frequency Domain - | MDF is a frequency at which the EMG |
| Median Power | power spectrum is divided into two |
| Frequency (MDF) | regions with equal amplitude. This is |
| | derived from the EMG sensor. |
| Total Power (TTP) | TTP is an aggregate of EMG power |
| | spectrum. This is derived from the |
| | EMG sensor. |
| Mean Power (MNP) | MNP is an average power of EMG |
| | power spectrum. This is derived |
| | from the EMG sensor. |
| Peak Frequency | PKF is a frequency at which the |
| (PKF) | maximum EMG power occurs. This is |
| | derived from the EMG sensor. |
| Spectral Moments | SM is an alternative statistical |
| (SM) | analysis way to extract feature |
| | from the power spectrum of EMG |
| | signal. This is derived from |
| | the EMG sensor. |
| Frequency ratio (FR) | FR is used to discriminate between |
| | relaxation and contraction of the |
| | muscle using a ratio between low- |
| | and high-frequency components of |
| | EMG signal. This is derived from |
| | the EMG sensor. |
| Power Spectrum | PSR is a ratio between the energy |
| Ratio (PSR) | P0 which is nearby the maximum |
| | value of EMG power spectrum and |
| | the energy P which is the whole |
| | energy of EMG power spectrum. This |
| | is derived from the EMG sensor. |
| Variance of Central | VCF is defined by using a number |
| Frequency (VCF) | of the spectral moments (SM0-SM2) |
| | and MNF. This is derived from |
| | the EMG sensor. |
| Motion | Consisting of a 3-axis accelerometer, |
| | 3-axis gyroscopeand 3-axis magnetometer, |
| | motion detection in 9 axis helps us to |
| | understand the exact orientation of a |
| | person. This understanding is made more |
| | accurate with the help of piezoelectric, |
| | force and vibration modules. This could |
| | be an analog, digital or optical sensor |
| | integrated with the wearable. |
| 3-axis accelerometer | The accelerometer measures earth's |
| | gravity field minus acceleration. |
| | This could be an analog, digital |
| | or optical sensor integrated with |
| | the wearable. |
| 3-axis gyroscope | The gyroscope sensor measures |
| | angular velocity. This could be |
| | an analog, digital or optical |
| | sensor integrated with the wearable. |
| 3-axis magnetometer | The magnetometer measures earth's |
| | magnetic field. This could be an |
| | analog, digital or optical sensor |
| | integrated with the wearable. |
| Piezo film | Monitors user motion and general |
| | activity level. This could be an |
| | analog sensor integrated with |
| | the wearable. |
| Piezo cable | Determines impact location and |
| | intensity during a person's fall. |
| | This could be an analog sensor |
| | integrated with the wearable. |
| Vibration | Shows presence or absence of |
| | discernible activity. This could be |
| | derived from a multitude of sensors. |
| Impact | The intensity with which the |
| | person has fallen or lost balance. |
| Altitude | Altitude is the distance above |
| | sea level. Areas are often |
| | considered “high-altitude” if |
| | they reach at least 2,400 meters |
| | (8,000 feet) into the atmosphere. |
| | This could be an analog sensor |
| | integrated with the wearable or it |
| | could be derived from a third-party |
| | application or device. |
| Weather condition | Weather condition is the meteorological |
| | day-to-day variations of the atmosphere |
| | and their effects on life and human |
| | activity. It includes temperature, |
| | pressure, humidity, clouds, wind, |
| | precipitation and fog. This could be |
| | an analog sensor integrated with the |
| | wearable or it could be derived from |
| | a third party application or device. |
| Barometric Pressure | Barometric pressure is the force |
| | exerted by the atmosphere at a |
| | given point. It is known as the |
| | “weight of the air”. This could |
| | be an analog sensor integrated |
| | with the wearable or it could be |
| | derived from a third party application |
| | or device. |
| Temperature | Atmospheric temperature is a |
| | measure of temperature at different |
| | levels of the Earth's atmosphere. |
| | It is governed by many factors, |
| | including incoming solar radiation, |
| | humidity and altitude. This could be |
| | an analog sensor integrated with the |
| | wearable or it could be derived from |
| | a third party application or device. |
| Humidity | Humidity is the amount of water |
| | vapor in the air. Too much or too |
| | little humidity can be dangerous. |
| | This could be an analog sensor |
| | integrated with the wearable or it |
| | could be derived from a third party |
| | application or device. |
| Video camera | A video camera connected with the |
| | device can help in taking video of |
| | the event. Such video frames can be |
| | analysed later to understand the |
| | frequency of seizures. |
| Microphone | A microphone connected to the device |
| | helps pick up relevant sounds from |
| | the person wearing our device, which |
| | can then later be used for analysis |

Input may also be obtained from third party IoT systems and applications also referred to as distributed IoT systems. Several IoT systems and applications allow data sharing among them. For example, the present subject matter can selectively access sensor data shared via Fitbit™ Application Programming Interface (API) to learn about users' activities, or access Smartcar™'s Connected Car API to securely communicate with vehicles, or use Netatmo™ Connect API programs to access customized weather services. Such additional information in the context of users from connected devices helps a better understanding of users and the environments in which they operate.

Another input may be obtained from third-party enterprise systems. Third-party enterprise systems and applications also provide detailed information about users and their health. Some examples of such third-party enterprise systems that the present subject matter can access include hospital management systems, diagnostic systems (EEG, MRI ECG, and the like), insurance systems, government databases, and the like, which are legally allowed to share and receive on demand medical information. Global positioning systems (GPS) can be used to understand locations frequented by users and this information can be used in context with the physiological data obtained by sensors to understand users' moods and health states with respect to a particular location.

Yet another input may be obtained from social media systems. Behavioral analysis of social media feeds of users from channels, such as, Facebook, Twitter, Instagram, and the like, provide indicators of health, especially the mental health of users. For example, a sentiment analysis of the tweets posted by a particular user or their peer group can be performed using machine learning classifiers (Max Entropy, Random Forest, and the like) to detect positive, negative, or neutral tweets, taking into account bigrams, URLs, hash tags, usernames, and emoticons. For example, a user may share that they had a bad day at work or that they are agitated about something. The ML program on learning of such an event co-relates this negative sentiment with physiological signals to assess the actual stress level of the user and suggest an intervention like a breathing exercise and the like to bring the stress level down. Such information can be mined from the social media profiles for the users.

Another input may be provided by the user. Demographic and psychographic information provided by users through a graphical user interface (GUI), for example, through a computing device or a wearable device is also a source for building a comprehensive profile of users. User input could also be sought in the form of interactions, feedback, ratings, and reviews. Medically relevant health assessment scales, such as, PHQ-9 (Patient Health Questionnaire), Stanford Health Assessment Questionnaire, and the like, may be implemented as part of the wearable device, which will provide unique, self-reported information on users' health. Such insights are used to correlate with the collective insights from other input sources to finetune or challenge the findings of the algorithms implemented in the wearable device.

The types of inputs mentioned above may be used in various combinations during the implementation of the present subject matter.

Using the inputs, or various combinations thereof, dynamic profiles of each individual are created by using artificial intelligence (AI)-based algorithms that are, for example, part of the processing unit of the wearable device or in the IoT network. Every individual user profile is characterized by some unique and some common signal patterns. Patterns are formed by combining two or more physiological signals, parameters, thresholds, climatic conditions, changes to locations, motion, and also from unique audio and video input recorded by the wearable device. These patterns are formed under different conditions and under varying circumstances, and may occur in response to or without internal and external stimulus. Owing to these factors every individual has unique signature patterns that may be analyzed to predict an adverse event before it occurs.

The different types of patterns that may be identified include normal patterns, abnormal patterns, disorder specific patterns, and unknown patterns.

Normal patterns are the patterns formed using physiological data pertaining to normal daily activities detected by the wearable device and recorded regularly from the individual in his or her normal surroundings, whether it is home or at work. These patterns could relate to activities, such as, talking, sitting, walking, sleeping, running, driving, cycling, and so on.

Abnormal patterns are the patterns that do not correspond to the normal recorded patterns of the individual. Such patterns may or may not indicate an adverse event. They may result from noise or could also imply an impact of the wearable device with a solid surface, and other such events that are not health related.

Disorder-specific patterns are the physiological patterns that correspond to a specific disorder, such as epilepsy, where two or more signals or parameters show interconnections and co-relation with each other. Such patterns may be based on environmental and location-related information in addition to physiological parameters that are together known to cause an adverse impact on the individual.

Unknown patterns are the patterns that are not hitherto seen in the individual.

The present subject matter also uses medically approved thresholds, logic, and rules to derive meaning from the different patterns. Thresholds refer to the upper and lower boundary limits around which the physiological parameters operate. Further, the thresholds set for an individual as determined over a period of time may or may not fall within a medically-acceptable range of values.

Although there are medically approved thresholds of physiological and biomedical signals, the AI system running in the wearable device's processor, either within or outside it, can identify an individual's threshold and dynamically adjust it. For example, the normal body temperature of an average individual is 98.6° F. If the temperature remains constantly at 97° F., it may not mean the individual is sick, rather his or her temperature threshold is lower than average. Such automatic thresholding reduces the risks of false alarms.

The individual profile and related patterns can be used to understand the health risks of groups of individuals with similar health issues. Such signal patterns can be used for risk classification and learning about the range of a particular disorder. AI-based learning helps in understanding how a particular disorder evolves over a period of time and what could be done to diminish or slow the progress of the disorder. The AI-based insights show: (a) the disorder at a particular phase of evolution, from low grade to severe, (b) triggers, stressors, and effects of medication and therapy, and (c) any underlying health conditions, both physical and mental that develop over a period of time.

The combination of profiles and corresponding patterns help to visualize unique disorder characteristics in individuals, identify underlying issues, classify the severity of disorders, identify propensity to risks, and predict events based on risks. Personalized profiles created using the present device provide an understanding about the physical and mental health of individuals, including helping to identify triggers and stressors impacting different chronic conditions. Profiles with similar patterns and characteristics can be grouped to study the evolution and progression of a disease or disorder. Such information can be used to reclassify disease and disorder types and provide better understanding to doctors.

The patterns are further processed to identify (a) stressors, (b) triggers, (c) reactions, and (d) recovery. Stressors refer to all patterns that indicate what causes stress in individuals. Triggers refer to patterns that show what led to a chain of events. Unlike stressors, they indicate positive events as well. Reactions refer to the patterns that indicate the events that are seen after a negative or a positive event. Recovery refers to the patterns that show what activities reverse a negative pattern.

Based on the various inputs and analysis, the device of the present subject matter can determine the physical and mental health of individuals remotely and provide faster access to emergency care based on real-time monitoring. Accordingly, the device of the present subject matter can be implemented for prediction and management of chronic disorders not limited to central nervous system (CNS) related disorders, cardiological disorders, psychological disorders, and orthopedic disorders. The device of the present subject matter can also be used in sports medicine, rehabilitation, physiotherapy, fitness, and wellness-related devices, not limited to the disorders, diseases, or conditions mentioned herein.

In an embodiment, a wearable device is embedded with a plurality of sensors, which may extract a large and continuous stream of data from an individual and the extracted data may be processed by a processing unit of the wearable device, to detect an adverse event.

In another embodiment, the extracted data may be communicated to a processing unit that is not a part of the wearable device. Based on the processing, the processing unit may initiate a chain of events, including informing other connected IoT systems automatically about the incoming adverse event, mobilizing care by alerting hospitals, caregivers, or other interested parties, providing alerts to concerned individuals in their preferred medium, and initiating steps to ensure safety of concerned individuals, before occurrence of an adverse event. Further, raw data and processed data from the processing unit may be communicated to a cloud environment on a continuous or scheduled basis.

The data extracted by the sensors undergo preprocessing, noise cancelation, filtering, and smoothening, before being further processed in the device processor, at a gateway device, other IoT device, a system, or in the cloud environment.

The present device uses one or more machine learning (ML) methods and AI techniques to create dynamic health profiles of individual users, to provide predictions and provide a basis to a decision support system, which is used by doctors or healthcare professionals for decision making.

Aspects of the present subject matter are further described in conjunction with appended Figures. It should be noted that the description and figures merely illustrate the principles of the present subject matter. It will thus be appreciated that various arrangements that embody the principles of the present subject matter, although not explicitly described or shown herein, can be devised from the description and are included within its scope. Moreover, all statements herein reciting principles, aspects, and implementations of the present subject matter, as well as specific examples thereof, are intended to encompass equivalents thereof.

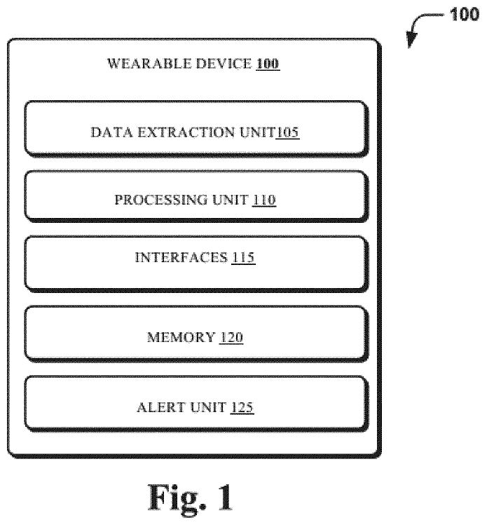

FIG. 1 illustrates a block diagram of a wearable device embedded with a plurality of sensors for prediction and management of chronic disorders, in accordance with an example embodiment of the present subject matter. In an embodiment, the wearable device 100 may include a data extraction unit 105 embedded within the device 100, a processing unit 110, communication interfaces 115, a storage unit 120, and an alert unit 125. In another embodiment, the wearable device 100 may include the data extraction unit 105 and the processing unit 110, and the alert unit 125 may be separate individual components connected to the data extraction 105 unit via any one of the communication mediums, such as, Wi-Fi, Bluetooth, 3F, 4G, and the like. It will be understood that the wearable device 100 also has batteries, a display unit, and sensors, though not explicitly shown herein.

In an embodiment, the wearable device 100 may be configured to be worn, by an individual whose health condition has to be predicted and managed, for example, as a glove, a sock, an arm band, vest, strap, or have any form factor that allows it to obtain the required information from the individual wearing the wearable device 100. The wearable device 100 may be embedded with a plurality of sensors having bio-medical sensors and climatic condition related sensors.

The bio-medical sensors may collect physiological signals and sense data related to the health of the individual. By way of an example, and not limitation, the bio-medical sensors include temperature sensors to sense skin temperature and body temperature sensor, respiration sensor, blood pressure sensor, electrodermal activity (EDA) sensor, electromyography (EMG) sensor, piezoelectricity sensor, accelerometer, gyroscope, magnetometer, pressure sensor, vibration sensor, electrocardiogram, and pulse oximetry. Further, the climatic condition related sensors may include, for example, pressure sensor, altitude sensor, temperature sensor, and humidity sensor. the climatic conditions can also be obtained from other IoT devices and systems that may provide the inputs to the wearable device 100. The wearable device 100 may include data related to different types of patterns, threshold, insights, recommendations, and other values along with health history and medication schedule of an individual.

Further, in an embodiment, the wearable device 100 may be provided with a global positioning system (GPS) to track a location of the individual wearing the wearable device 100 and accordingly the location of the individual may be communicated to caregivers or healthcare professionals for immediate action. In another example, the GPS information may be obtained, for example, from a mobile device associated with the individual.

In an embodiment, the processing unit 110 may include one or more processors coupled to the storage unit 120. The processing unit 110 may be personalized for every individual wearing the wearable device 100. The storage unit 120 is referred to as a memory, including any device known in the art including, for example, volatile memory, such as, static random access memory (SRAM) and dynamic random access memory (DRAM), and/or non-volatile memory, such as read only memory (ROM) and erasable programmable ROM. The storage unit 120 may include data related to different types of patterns and threshold values along with health history and medication schedule of an individual. For instance, different types of patterns include normal patterns, abnormal patterns, disorder specific patterns, and unknown patterns. Further, the communication interface(s) 115 can facilitate multiple communications within a wide variety of networks and protocol types, including wired networks, for example, local area network (LAN), cable, and the like; and wireless networks, such as, wireless LAN (WLAN), cellular, or satellite. For the purpose, the interface(s) may include one or more ports for connecting a number of devices to each other or to another computing system.

The processing unit 110 receives data related to the above-mentioned parameters and processes the data for detection of adverse events. The data may be used for the prediction of several types of disorders prior to their occurrence. For instance, in the case of epilepsy, the wearable device can provide a prediction before a seizure based on the auras that an individual might experience prior to attack. The predictions may vary from individual to individual, as certain auras are not detectible, while some individuals do not have any auras before the attack. Further, several disorders, especially those related to epilepsy or cardiological events, accompanies a particular level of electrodermal activity (EDA) or heart rate (HR), which can be correlated to the onset of the attack.

In an embodiment, the predictions that the wearable device 100 provides is based on several factors including a statistical relationship between the sensor data and the signal thresholds. The processing unit 110 performs edge analytics on the received data from the data extraction unit 105 to predict adverse events based on the signal thresholds, before the occurrence of the adverse events. For instance, the adverse events include epileptic attack or a stroke. Edge analytics refers to a method of data analysis that is performed using advanced computing technologies, instead of sending the data to a centralized computing unit for processing. This facility saves time and enables onboard processors to provide insights about the data instantaneously. In particular, one or more analytical computational methods executed by the processing unit 110 receives patterns formed of the sensed data as an input and breaches to threshold values to predict the health status of the individual.

In one embodiment of the present subject matter, the sensor data may be processed using an appropriate software to obtain a score for an individual, which can then be used to generate a pattern. Pre-processing of the data is done, including converting analog signals to digital, converting multiple formats to a consistent and acceptable format, filtering and smoothening of signals, and preparing signals for input into algorithms. In an example, a Butterworth filter is applied to filter a raw blood volume pulse (BVP) data. Depending upon the situation or context, the right set of algorithms are selected to ascertain the stress level and derive a health score for an individual.

As an example of one embodiment of the present subject matter, for calculating the stress score of a person at rest, the input sources used are galvanic skin conductance (GSR) and heart rate (HR) from device sensors, environmental data, such as, temperature and humidity, from third-party APIs, accelerometer data from a mobile phone, and user input data. Filtered GSR values are obtained from the sensor and a score is derived using a computation. For obtaining a HR score, filtered HR values are obtained from sensors, and compared to medically approved thresholds determined by age and gender. The age and gender are a user input stored within the system. A total score is calculated using all the above, i.e., GSR score, HR score, and motion score.

Such scores may be collected for an individual continuously for over a period of time, generating patterns. An example pattern for an individual is shown in Table 2. A flow of events is observed; how the individual started with having medium level of stress, which became high, then critical, came back to medium and then to low. So, the period from 10 a.m. to 1 p.m. was very stressful for the individual; he slowly recovered and then came back to normal. The gap between the critical phase and the recovery phase is not much, whereas the stress buildup happened over a period of time. This indicates that the person went through certain situations that induced a high level of stress. This could also lead to an event like a seizure.

TABLE 2
Flow of Events for Change in Pattern for an Individual

| | | | HRV | | | | | | | |
|---|---|---|---|---|---|---|---|---|---|---|
| | HRV | HRV | LF/HF | | GSR | GSR | Mobility | Location | Stress | |
| Time | SDNN | RMSSD | Ratio | HRV TP | Min | Max | Status | Label | Level | Score |
| 10:00:00 | Normal | Normal | Low | Low | 2 | 2.5 | Active | Home | Medium | 62% |
| 10:50:00 | Normal | Normal | Normal | Low | 2.5 | 4 | Active | Home | Medium | 52% |
| 11:10:00 | Normal | Normal | Normal | Low | 2.5 | 4 | Active | In Transit | Medium | 51% |
| 11:20:00 | Normal | High | Low | High | 2.5 | 6 | Active | In Transit | High | 49% |
| 11:30:00 | Normal | High | Low | High | 2.5 | 6.6 | Active | Work | High | 49% |
| 13:00:00 | High | High | Low | High | 2.5 | 10.4 | Active | Work | High | 28% |
| 13:20:00 | High | High | Low | High | 2.5 | 10.6 | Active | Work | Critical | 18% |
| 13:30:00 | High | High | Low | High | 2.5 | 12 | Active | Work | Critical | 17.20% |
| 13:40:00 | High | High | Low | High | 2.5 | 12 | Active | Park | Critical | 18.00% |
| 13:50:00 | Normal | Normal | Normal | Low | 2 | 6.1 | Rest | Park | Medium | 33% |
| 14:00:00 | Normal | Normal | Normal | Low | 2 | 6 | Rest | Park | Medium | 42% |
| 14:10:00 | Normal | Normal | Normal | Normal | 2 | 6 | Rest | Park | Low | 70% |
| 15:00:00 | Normal | Normal | Normal | Normal | 2 | 2.5 | Rest | Park | Low | 74% |

When that event happened, some intervention was given and that is indicative of the person's stress level becoming lower. If this pattern is repeated over a period of time, an alert can be provided (as soon as the person gets through medium level of stress) saying that based on previous evidence the individual may be having a seizure if the stress goes below 45 or 50%.

It could also be inferred that some kind of intervention happened, for e.g., a medicine was given to that person that helped bring down his stress. If the medicine or intervention is working, then the stress level will come down and the person will recover. If the medicine is not working, then the stress level will continue to be higher. This may lead to further deterioration of health and could also lead to a fatality.

This kind of pattern-building and scoring helps in understanding how a disease or a disorder evolves, how much time is available to provide predictions so that a negative event is averted, what kind of interventions are more successful, what works, and what does not work for an individual.

When an adverse event is detected by the processing unit 110, the processing unit 110 may communicate to the alert unit 125, which may directly inform caregivers, doctors, and hospitals about the current health state of the individual wearing the wearable device 100. Such measures enable proactive care to be provided to individuals in need.

For example, when an unseen pattern is observed, the processing unit 110 checks immediately for the existence of the pattern in a patterns repository within the device, in a cloud environment or a server computing device. If it exists and is previously known to the system, two actions are triggered, i.e., the processing unit 110 is notified of the existence of the pattern and the processing unit 110 is instructed to keep a tab on the new pattern. If it occurs repeatedly, then after a predefined number of times, it is moved to the disorder-specific patterns store in the storage unit 120.

Thus, the wearable device 100 can predict an occurrence of the adverse events based on sensor data to prevent any harm that could come to individual because of delayed care.

In another embodiment, as previously discussed, the processing unit 110 may be a separate component communicatively connected with the wearable device via one of communication mediums, such as, Bluetooth, Wi-Fi, 3G, 4G, and the like. The placement of processing unit 110 whether it is within the wearable device or outside the wearable device 100 depends on several factors such as processing power battery requirement of different sensors, data transmission capabilities, and the like.

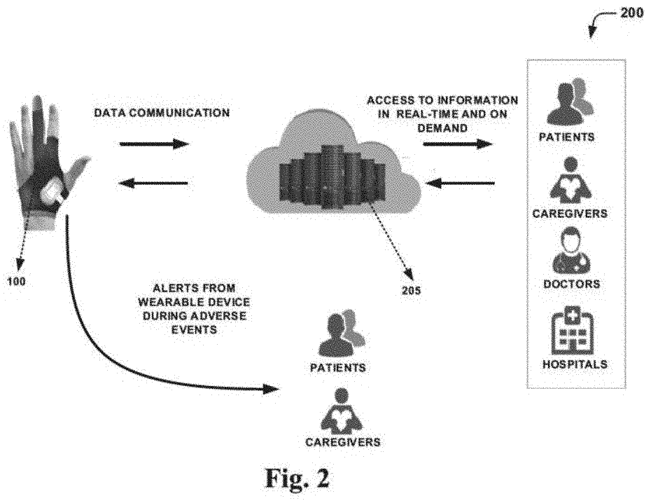

FIG. 2 illustrates a health prediction system implemented with the wearable device of the FIG. 1, in accordance with an example embodiment of the present subject matter. As shown in FIG. 2, the wearable device 100 worn by an individual may be communicatively connected to a cloud computing platform 205 or a server computing device 205 via one of communication mediums, such as, Wi-Fi, Bluetooth, 3G, 4G, and the like. Although the system 200 is shown with the one wearable device 100 connecting the cloud computing platform 205, it is understood to a person skilled in the art that multiple wearable devices worn by multiple individuals may be communicatively connected to the cloud computing platform 205 or the server computing device 205. The cloud computing platform 205 or the server computing device 205 includes one or more processors, memory, an input unit, such as, a key board and/mouse, and an output unit, such as, a display (not shown in the FIG. 2). In an aspect, different patterns such as normal patterns, abnormal patterns, disorder-specific patterns, unknown patterns, threshold values, and one or more artificial intelligence computational method instructions are stored in the memory.

The cloud computing platform 205 is a decision support system and is based on a combination of a medical knowledge base and streaming data of individuals wearing the wearable device 100. The cloud computing platform 205 may receive raw sensor data and processed sensor data from different wearable devices 100 connected to the cloud computing platform 205 on a continuous or scheduled basis. The cloud computing platform 205 further processes the received data using the one or more AI techniques and machine learning methods to provide insights to doctors automatically. For instance, different types of data analysis and comparisons are performed at the cloud computing platform 205 to obtain different insights. Filtered and curated information in the cloud storage forms the basis of the decision support system. The decision support system may facilitate the healthcare professionals or the doctors to come up with customized management plans for his or her patients or the individuals.

The cloud computing platform 205 can identify unique characteristics of every individual, which is otherwise not discernible without continuous long-term monitoring. Such measures help healthcare professionals or doctors to learn about previously unknown characteristics of different types of disorders both in the individual or his or her peer group, compare individuals in different peer groups, and obtain information about the effects of different types of medicines among different individuals and groups. Further, the decision support system may help doctors or healthcare professionals recommend treatment plans and provide notification about any future adverse events related to individuals wearing the device.

In an example use of the present subject matter, Table 3a, 3b, 3c shows the patterns generated for several individuals before, during, and after a seizure. The data is generated using heart rate variability features. In the pre-ictal period, individuals show a variety of patterns. However, during ictal, several individuals display similar patterns. In the post-ictal period, individual patterns may be similar or different.

TABLE 3a
Example Patterns Pre-Ictal Period

| Patient | | | | | LF/HF | |
|---|---|---|---|---|---|---|
| ID | SDNN | RMSSD | LF | HF | Ratio | TP |
| 1 | Low | Normal | Low | Low | Low | Low |
| 2 | Low | High | Low | Low | Low | Low |
| 3 | Low | High | Low | High | Low | High |
| 4 | Low | Normal | Low | Low | Low | Normal |
| 5 | Low | Normal | Low | Low | Low | Low |
| 6 | Low | Normal | Low | Low | Low | Low |
| 7 | Low | Normal | Low | Low | Normal | Low |
| 8 | Low | Normal | Normal | Low | High | Normal |
| 9 | Normal | High | Low | Normal | Low | Normal |
| 10 | Normal | High | High | High | Low | High |
| 11 | Normal | High | Normal | High | Low | High |
| 12 | Low | High | Low | High | Low | Low |
| 13 | Low | High | Low | Low | High | Low |
| 14 | Normal | High | Normal | Normal | Normal | High |
| 15 | Low | Normal | Low | Low | Normal | Low |

TABLE 3b
Example Patterns Ictal Period

| Patient | | | | | LF/HF | |
|---|---|---|---|---|---|---|
| ID | SDNN | RMSSD | LF | HF | Ratio | TP |
| 1 | Normal | High | High | High | Low | High |
| 2 | Normal | High | Low | High | Low | High |
| 3 | Normal | High | Low | High | Low | High |
| 4 | Normal | High | Normal | Low | Normal | High |
| 5 | Normal | High | High | High | Low | High |
| 6 | Normal | High | High | High | Low | High |
| 7 | Normal | High | High | High | Low | High |
| 8 | Normal | High | High | High | Low | High |
| 9 | Normal | High | High | High | Low | High |
| 10 | Normal | High | High | High | Low | High |
| 11 | Normal | High | High | High | Low | High |
| 12 | Normal | High | High | High | Low | High |
| 13 | Normal | High | High | High | Low | High |
| 14 | Normal | High | High | High | Low | High |
| 15 | Normal | High | High | High | Low | High |

TABLE 3c
Example Patterns Post Ictal Period

| Patient | | | | | LF/HF | |
|---|---|---|---|---|---|---|
| ID | SDNN | RMSSD | LF | HF | Ratio | TP |
| 1 | High | High | Low | Low | Low | High |
| 2 | Normal | High | High | High | Low | High |
| 3 | Normal | High | High | High | Low | High |
| 4 | Normal | High | High | High | Low | High |
| 5 | Low | High | Low | High | Low | High |
| 6 | Normal | High | High | High | Low | High |
| 7 | High | High | High | High | Low | High |
| 8 | Normal | High | High | High | Low | High |
| 9 | Normal | High | High | High | Low | High |
| 10 | Normal | High | High | High | Low | High |
| 11 | Normal | High | High | High | Low | High |
| 12 | Normal | High | High | High | Low | High |
| 13 | Normal | High | High | High | Low | High |
| 14 | Normal | High | High | High | Low | High |
| 15 | High | High | High | High | Low | High |

Patterns derived from physiological signals can be very different for each individual, but as a response to stress, individuals may end up having similar kinds of patterns, indicative of a negative health outcome. Patterns help to understand group behavior. People with similar kinds of patterns during the pre-ictal, ictal, or post-ictal period can be grouped into different classes and their stressors, triggers, reactions, and recovery can be studied to understand what medications and therapies work for individuals in a group.

As shown in FIG. 2, patients or individuals, caregivers, doctors, and hospitals can access information from the cloud computing platform 205 using user-specific applications and dashboards through any form of an internet enabled device, including, but not limited to, mobile phones, smart watches, desktops, and the like.

Further, the cloud computing platform 205 has a mechanism to identify each individual such that the insights that are relevant to the corresponding individual may be stored in separate units.

Further, the decision support system may be helpful for the doctors to understand how disorders evolve in an individual over a particular period and its impact on a group of people. Doctors can classify and reclassify the individuals based on evidence streaming from the individual's body. Further, the decision support system facilitates doctors to study the efficacy of drugs and see how a particular drug is working on the individual or patient. Further, the doctors can make a choice regarding assessing an individual for surgery and risk of adverse circumstances like sudden unexpected death in epilepsy (SUDEP). The decision support system is also useful for non-specialist doctors, who can learn how specialists treat particular cases, and they can emulate their method. In addition, the decision support system helps in understanding how moods, auras, sleep, and medicines are interrelated and how they can lead to adverse effects.

The wearable device 100 and the system 200 of the present subject matter is capable of providing notices and alerts, remote consulting, consultation with or without internet, and long term remote monitoring.

In an embodiment, the wearable device 100 is capable of providing preventive measures when an individual is about to have an adverse event. As previously discussed, when the wearable device 100 predicts the onset of an attack, caregivers are notified and asked to take preventive measures before the secondary symptoms of the attack, like abnormal heart rate, can harm the individual. The device 100 assists in taking proactive action by doctors or caregivers to abort an attack if possible or to provide immediate assistance on the onset of the attack. This device also helps in tracking a person using GPS and providing notifications to caregivers in case of an alarm.

Remote consulting services enable an individual to obtain medical advice from healthcare practitioners and specialists irrespective of the geography or location of the individual. The remote consulting system that comes as part of the present system enables individuals even in remote areas to access healthcare professionals anywhere in the world and stream real-time health data for immediate access to care. This service could be accessed via any internet enabled device, and using this interface doctors and their patients can have video communication and real-time health data streaming simultaneously.

The present device 100 also works without internet. If an individual does not have access to internet (e.g., in a remote village scenario), all data belonging to the individual gets saved on the server 205 and can be downloaded anytime and given to a doctor.

Continuous monitoring of an individual with a chronic disorder plays a very important role in arriving at a correct diagnosis. It could lead to timely preventive measures and provide important insights for appropriate treatment. The system 200 of the present subject allows long term remote monitoring of individuals from the comforts of their homes and in their natural surroundings by healthcare professionals in a simple and portable form factor. Long term and remote monitoring with the device allows healthcare professionals to get intrinsic information about an individual like efficacy of medicines, triggers, frequency of adverse events, moods, sleep patterns, and so on. With access to relevant and curated information about every individual wearing the device, healthcare professionals can get required insights to provide a customized management plan designed for their specific case.

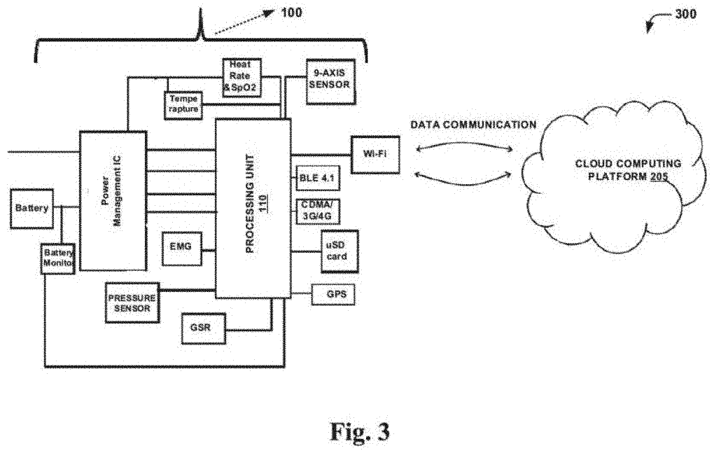

FIG. 3 illustrates a block diagram of the health prediction system of FIG. 2, in accordance with an example embodiment of the present subject matter. Different components of FIG. 3 show how the components of the system 300 interact with each other. Different components of the wearable device 100 are connected to a processing unit 110 or a micro controller of the wearable device 100. Further, the wearable device 100 includes a power management IC. The different components, include, but are not limited to, communication interfaces such as Bluetooth and Wi-Fi; and sensors such as a 9-axis sensor, barometric pressure sensor, temperature sensor, and EMG sensor, as shown in FIG. 3. Further, 30 the wearable device 100 is communicatively connected to the cloud computing platform 205 as shown in FIG. 3.

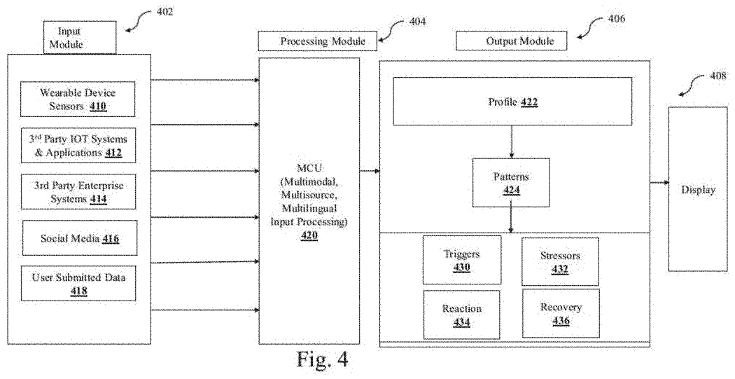

FIG. 4 illustrates another block diagram of the health prediction system of FIG. 2 implemented in an IoT network, in accordance with an example embodiment of the present subject matter. As shown in FIG. 4, Inputs 402 for an individual may be received from various sensors in the IoT network as discussed above. For example, wearable device sensors 410 may provide inputs related to physiological and environmental conditions of the individual, third party systems and applications 412 may provide inputs related to climatic conditions, environmental conditions, home conditions, etc., third party enterprise systems 414 may provide inputs related to medical records, etc., social media systems 416 may provide inputs related to sentiment analysis of the user, and user submitted data 418 may be received as input for example, from a web server, wearable device, computing device, and the like. The various inputs 402 are then processed at block 404. For example, a wearable device or a server in a cloud environment may receive the various inputs 402, store the inputs, and the processor therein may perform multi-modal, multi-source, and multi-lingual input processing 420. For example, the processing 420 may include creating a profile for the individual, identifying patterns, and predicting adverse events as discussed above. Based on the processing 420, outputs 406 may be generated. The outputs include the profile 422, the patterns, 424, the triggers 430, the stressors 432, the reaction 434, and the recovery 436 as mentioned above. These may be stored on the wearable device or computing device. Further, the outputs may be displayed on a display 408, for example, on the wearable device or the computing device. In one example, a report may also be generated and may be shared, for example, over email etc. Further, in case the processing 404 predicts an adverse event, an alert may b e generated as discussed above.

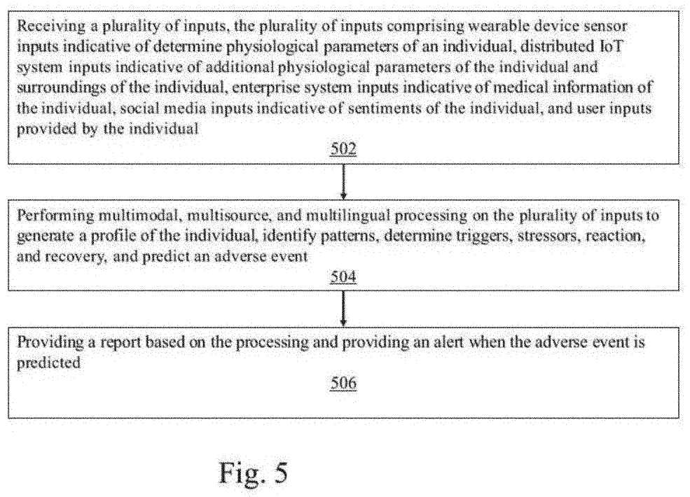

The methods used for health prediction will now be further described with reference to FIG. 5. While the method illustrated in FIG. 5 may be implemented in any system, for discussion, the method is described with reference to the implementations illustrated in FIG. 1-4

FIG. 5 illustrates an example process for health prediction, in accordance with an example embodiment of the present subject matter. At block 502, a plurality of inputs are received, the plurality of inputs comprising wearable device sensor inputs indicative of physiological parameters of an individual, distributed IoT system inputs indicative of additional physiological parameters of the individual and surroundings of the individual, enterprise system inputs indicative of medical information of the individual, social media inputs indicative of sentiments of the individual, and user inputs provided by the individual.

At block 504, multimodal, multisource, and multilingual processing is performed on the plurality of inputs to generate a profile of the individual, identify patterns, determine triggers, stressors, reaction, and recovery, and predict an 10 adverse event.

At block 506, a report is provided based on the processing and provide an alert when the adverse event is predicted.

Although embodiments for the wearable device and the IoT network with the wearable device have been described in language specific to structural features, it is to be understood that specific features are disclosed as example embodiments for implementing the claimed subject matter.

1. A computing system in an Internet of Things (IoT) network environment, the computing system being adapted for prediction and management of chronic disorders, the computing system comprising:
an input module to receive a plurality of inputs, the plurality of inputs comprising wearable device sensor inputs indicative of physiological parameters of an individual, distributed IoT system inputs indicative of additional physiological parameters of the individual and surroundings of the individual, enterprise system inputs indicative of medical information of the individual, social media inputs indicative of sentiments of the individual, and user inputs provided by the individual;
a processing module to perform multimodal, multisource, and multilingual processing on the plurality of inputs to generate a profile of the individual, identify patterns, determine triggers, stressors, reaction, and recovery, and predict an adverse event; and
a display module to provide a report based on the processing and provide an alert when the adverse event is predicted.

2. The computing system as claimed in claim 1, wherein the computing system is a wearable device.

3. The computing system as claimed in claim 1, wherein the physiological parameters and additional physiological parameters are selected from Skin temperature, Body temperature, Heart Rate (HR), Heart rate variability (HRV), Blood Pressure and trend, Respiration and trend, SpO2, Electrodermal Activity (EDA), Electromyography (EMG), Motion, 3-axis accelerometer, 3-axis gyroscope, 3-axis magnetometer, Piezo film, Piezo cable, Vibration, Impact, Altitude, and combinations thereof.

4. The computing system as claimed in claim 1, wherein the parameters indicative of surroundings of the individual include user activity, vehicle information, weather information, and audio and video input.

5. The computing system as claimed in claim 1, wherein the enterprise system inputs indicative of medical information of the individual include hospital data, diagnostics data, and insurance data.

6. The computing system as claimed in claim 1, wherein the user inputs provided by the individual include responses to health questionnaires, feedback, and self-reported information.

7. The computing system as claimed in claim 1, wherein the processing module is to identify patterns based on combining two or more physiological parameters, threshold, climatic conditions, changes to locations, motion, and audio and video input.

8. The computing system as claimed in claim 7, wherein the patterns include normal patterns, abnormal patterns, disorder specific patterns, and unknown patterns.

9. A method for prediction and management of chronic disorders in an IoT network environment, the method comprising:
receiving a plurality of inputs, the plurality of inputs comprising wearable device sensor inputs indicative of physiological parameters of an individual, distributed IoT system inputs indicative of additional physiological parameters of the individual and surroundings of the individual, enterprise system inputs indicative of medical information of the individual, social media inputs indicative of sentiments of the individual, and user inputs provided by the individual;
performing multimodal, multisource, and multilingual processing on the plurality of inputs to generate a profile of the individual, identify patterns, determine triggers, stressors, reaction, and recovery, and predict an adverse event; and providing a report based on the processing and providing an alert when the adverse event is predicted.

10. The method as claimed in claim 9, wherein the physiological parameters and additional physiological parameters are selected from Skin temperature, Body temperature, Heart Rate (HR), Heart rate variability (HRV), Blood Pressure and trend, Respiration and trend, SpO2, Electrodermal Activity (EDA), Electromyography (EMG), Motion, 3-axis accelerometer, 3-axis gyroscope, 3-axis magnetometer, Piezo film, Piezo cable, Vibration, Impact, Altitude, and combinations thereof.

11. The method as claimed in claim 9, wherein the parameters indicative of surroundings of the individual include user activity, vehicle information, weather information, and audio and video input.

12. The method as claimed in claim 9, wherein the enterprise system inputs indicative of medical information of the individual include hospital data, diagnostics data, and insurance data.

13. The method as claimed in claim 9, wherein the user inputs provided by the individual include responses to health questionnaires, feedback, and self-reported information.

14. The method as claimed in claim 9, wherein the processing module is to identify patterns based on combining two or more physiological parameters, thresholds, climatic conditions, changes to locations, motion, and audio and video input.

15. The method as claimed in claim 9, wherein the patterns include normal patterns, abnormal patterns, disorder specific patterns, and unknown patterns.

16. The method as claimed in claim 9, comprising preprocessing the plurality of inputs including performing noise cancelation, filtering, and smoothening.

17. The method as claimed in claim 9, comprising performing AI-based learning based on patterns of group of individuals with similar patterns to identify evolution of a disorder over a period of time from low grade to severe, correlate triggers, stressors, and effects of medication and therapy, and track underlying physical and mental health conditions that develop over a period of time.

18. The method as claimed in claim 9, wherein predicting an adverse event comprises calculating a stress score of the individual based on sensor data, IoT application and network data, enterprise data, social media data and user input.
